SEQUENCE LISTING (1 sequence)

SEQ ID NO: 1 - DNA, 18 bp, Artificial Sequence
  Bcl-2 Antisense Oligomer
tctcccagcgtgcgccat

(19) United States
(12) Patent Application Publication
(10) Pub. No.: US 2008/0171718 A1
(43) Pub. Date: Jul. 17, 2008

(54) Methods and Compositions for Treating Cancer Using BCL-2 Antisense Oligomers, Tyrosine Kinase Inhibitors, and Chemotherapeutic Agents

(76) Inventor: Bob D. Brown, Millington, NJ (US)

Correspondence Address: DIEHL SERVILLA LLC, 77 BRANT AVE, SUITE 210, CLARK, NJ, 07066, US

(21) Appl. No.: 11/935,654

(22) Filed: Nov. 6, 2007

Related U.S. Application Data
(60) Provisional application No. 60/864,859, filed on Nov. 8, 2006.

(51) Int. Cl. A61K 31/7052; C12N 9/99; A61P 35/00

(52) U.S. Cl. 514/44; 435/184

(57) ABSTRACT

Methods and compositions are provided for treating cell-proliferative related disorders such as cancer. Methods of inhibiting the growth of cancer cells comprise contacting the cancer cells with a Bcl-2 antisense oligomer; contacting the cancer cells with a tyrosine kinase inhibitor; and contacting the cancer cells with a cytotoxic chemotherapeutic agent. Methods of treating cancer in a human comprise administering to the human a Bcl-2 antisense oligomer, a tyrosine kinase inhibitor, and a cytotoxic chemotherapeutic agent. Kits containing compositions in amounts sufficient for at least one cycle of treatment comprise a triplet combination therapy of a Bcl-2 antisense oligomer, a tyrosine kinase inhibitor, and a cytotoxic chemotherapeutic agent. In selected embodiments, the tyrosine kinase inhibitor is one that targets cell surface kinase receptors, such as VEGFR (e.g., VEGFR1, VEGFR2, VEGFR3), PDGFR, KIT, and FLT-3.

### CROSS-REFERENCE TO RELATED APPLICATION

The application claims the benefit of and priority to U.S. Provisional Patent Application Ser. No. 60/864,859, filed on Nov. 8, 2006, which is hereby incorporated by reference.

### FIELD

The present invention relates to methods of treating cancer, and compositions for doing the same, which comprise inhibiting the expression of a Bcl-2 protein and a tyrosine kinase in conjunction with administering cytotoxic chemotherapeutic agents. Specifically, the tyrosine kinase inhibitor is one that targets cell surface kinase receptors, such as VEGFR (e.g., VEGFR1, VEGFR2, VEGFR3), PDGFR, KIT, and FLT-3.

### BACKGROUND

Recent strategies for treating cancer have included developing agents capable of regulating certain cell processes, such as cell proliferation, angiogenesis, and apoptosis. Traditional cytotoxic anticancer agents are designed to kill tumor cells by inducing apoptosis. Apoptosis is known to be inhibited by, for example, the Bcl-2 family of proteins. In turn, cytotoxic chemotherapy can be rendered less effective in the presence of an overexpression or imbalance of Bcl-2 proteins. Oblimersen sodium (G3139, Genasense®, Genta Incorporated, Berkeley Heights, N.J.) is an antisense oligonucleotide (AS-ON) that is designed to decrease Bcl-2. The efficacy of certain anticancer treatments using an apoptosis-modulating strategy has been enhanced with oblimersen Bcl-2 antisense therapy.

Because of their role in cellular signal transduction cascades, protein kinases have also become target classes for anticancer drug development. Several small molecule kinase inhibitors such as imatinib, gefitinib, and erlotinib have been approved for anticancer therapies. Receptor-type tyrosine kinases have a large number of transmembrane receptors with diverse biological activity. About twenty different subfamilies of receptor-type tyrosine kinases have been identified. Cell surface kinases include KIT (stem cell factor receptor), vascular endothelial growth factor receptors (VEGFR1, VEGFR2, and VEGFR2), and fms-like tyrosine kinase-3 (FLT3).

Several receptor-type tyrosine kinases, and the growth factors that bind thereto, have been suggested to play a role, directly or indirectly, in angiogenesis. One such receptor-type tyrosine kinase is vascular endothelial cell growth factor receptor 2 or VEGFR-2, since it binds VEGF with high affinity. Angiogenesis is characterized by excessive activity of vascular endothelial growth factor (VEGF).

Generally, an anticancer drug used in isolation cannot cure cancer alone. Often, the use of two or more drugs together offers a more effective alternative. There is a continuing need to provide anticancer combination therapies that are tailored to an individual patient's needs. There is also a continuing need to provide anticancer regimens that halt tumor growth, delay re-growth, and reduce the rate of re-growth.

### SUMMARY

Methods and compositions are provided for treating cell-proliferative related disorders such as cancer. In one aspect of the present invention, methods of inhibiting the growth of cancer cells comprise contacting the cancer cells with a Bcl-2 antisense oligomer; contacting the cancer cells with a tyrosine kinase inhibitor; and contacting the cancer cells with a cytotoxic chemotherapeutic agent. In one embodiment, the Bcl-2 antisense oligomer comprises an oblimersen compound. In another embodiment, the tyrosine kinase inhibitor targets a cell surface kinase receptor. The cell surface kinase receptors can include vascular endothelial growth factor receptors, stem cell factor receptors, and/or fms-like tyrosine kinase-3 receptors. In one embodiment, the cytotoxic chemotherapeutic agent comprises dacarbazine, docetaxel, paclitaxel, cisplatin, 5-fluorouracil, doxorubicin, etoposide, cyclophosphamide, fludarabine, irinotecan, or cytosine arabinoside (Ara-C).

In another aspect of the present invention, methods of treating cancer in a human comprise administering to the human a Bcl-2 antisense oligomer, a tyrosine kinase inhibitor, and a cytotoxic chemotherapeutic agent. In one embodiment, the Bcl-2 antisense oligomer is administered twice per week, the tyrosine kinase inhibitor is administered five times per week, and the cytotoxic chemotherapeutic agent is administered once per week. In another embodiment, the Bcl-2 antisense oligomer comprises an oblimersen compound; the tyrosine kinase inhibitor comprises sunitinib, sorafenib, or combinations thereof; and the cytotoxic chemotherapeutic agent comprises paclitaxel. In a further embodiment, the Bcl-2 antisense oligomer is administered after the cytotoxic chemotherapeutic agent in each cycle of treatment.

A further aspect of the present invention includes a kit comprising a Bcl-2 antisense oligomer in an amount sufficient for one 5-day cycle of cancer treatment, a tyrosine kinase inhibitor in an amount sufficient for one 5-day cycle of cancer treatment, and a cytotoxic chemotherapeutic agent in an amount sufficient for one 5-day cycle of cancer treatment.

### BRIEF DESCRIPTION OF THE FIGURES

The teachings of the present invention can be readily understood by considering the following detailed description in conjunction with the accompanying drawings, in which:

FIG. 1 shows tumor volume versus days (post implantation) for treatments in accordance with the present invention.

FIG. 2 shows tumor volume versus days (post implantation) for treatments in accordance with the present invention.

FIG. 3 shows tumor volume versus days (post implantation) for treatments in accordance with the present invention.

FIG. 4 shows tumor volume versus days (post implantation) for treatments in accordance with the present invention.

### DETAILED DESCRIPTION

Methods and compositions are provided for treating cell-proliferative related disorders such as cancer. In one aspect of the present invention, methods of inhibiting the growth of cancer cells comprise contacting the cancer cells with a Bcl-2 antisense oligomer; contacting the cancer cells with a tyrosine kinase inhibitor; and contacting the cancer cells with a cytotoxic chemotherapeutic agent. In another aspect of the present invention, methods of treating cancer in a human comprise administering to the human a Bcl-2 antisense oligomer, a tyrosine kinase inhibitor, and a cytotoxic chemotherapeutic agent. A further aspect of the present invention includes a kit comprising a Bcl-2 antisense oligomer in an amount sufficient for at least one cycle of cancer treatment, a tyrosine kinase inhibitor in an amount sufficient for at least one cycle of cancer treatment, and a cytotoxic chemotherapeutic agent in an amount sufficient for at least one cycle of cancer treatment.

Triplet therapy comprising a combination of a Bcl-2 antisense oligomer, a cytotoxic chemotherapeutic agent, and a tyrosine kinase inhibitor shows surprising results in suppressing and delaying tumor growth and reducing the rate of tumor regrowth after treatment is completed. In selected embodiments, the tyrosine kinase inhibitor is one that targets cell surface kinase receptors, such as VEGFR1, VEGFR2, VEGFR3, KIT, and FLT-3.

As used herein, the phrase “cell-proliferative disorder” refers to a condition marked by aberrant (e.g., uncontrolled) cell division. Such a disorder encompasses diseases involving cell division induced by, or concomitant with, for example, bacterial infections, viral infections, inflammation, inflammatory conditions (e.g., anaphylaxis, allergy, arthritis, asthma, microbial infection, parasitic infection), malignant cellular transformation or mutation, and autoimmune disorders.

As used herein, the term “cancer” describes a cell-proliferative disorder in which the transformation or mutation of a normal cell results in abnormal cell growth, which may be followed by an invasion of adjacent tissues by these abnormal cells, and which may also be followed by lymphatic, cerebral spinal fluid, or blood-borne spread of these abnormal cells to regional lymph nodes and/or distant sites, i.e., metastasis.

As used herein, the term “tumor” or “growth” means increased tissue mass, which includes greater cell numbers as a result of faster cell division and/or slower rates of cell death. Tumors may be malignant or non-malignant cancers.

As used herein, the phrases “treating cancer” and “treatment of cancer” mean to inhibit the replication of cancer cells, inhibit the spread of cancer, decrease tumor size, lessen or reduce the number of cancerous cells in the body, or ameliorate or alleviate the symptoms of the disease caused by the cancer. The treatment is considered therapeutic if there is a decrease in mortality and/or morbidity, or a decrease in disease burden manifest by reduced numbers of malignant cells in the body.

As used herein, the term “cycle” and the phrase “cycle of therapy” mean a period of time during which treatment is administered to the patient. Typically, in cancer therapy a cycle of therapy is followed by a rest period during which no treatment is given. Following the rest period, one or more further cycles of therapy may be administered, each followed by additional rest periods.

### Bcl-2 Antisense Oligomer

A Bcl-2 antisense oligomer refers to an oligomer that hybridizes to a Bcl-2 mRNA or pre-mRNA. Also encompassed are oligomers that hybridize to a portion of a Bcl-2 mRNA or pre-mRNA. Such oligomers may be capable of decreasing translation of the Bcl-2 message. Accordingly, the invention contemplates use of one or more Bcl-2 antisense oligomers, or a derivative, analog or fragment thereof. As used herein, the term “derivative” refers to any pharmaceutically acceptable homolog, analogue, or fragment, which retains the ability to bind to a Bcl-2 mRNA or a portion thereof. Antisense oligomers suitable for use in the invention include oligomers which range in size from 5 to 10, 10 to 20, 20 to 50, 50 to 75, 75 to 100, or 101 to 1000 bases in length; preferably 10 to 40 bases in length; more preferably 12 to 25 bases in length; most preferably 18 bases in length.

The target sequences may be RNA or DNA, and may be single-stranded or double-stranded. Target molecules include, but are not limited to, pre-mRNA, mRNA, and DNA. In one embodiment, the target molecule is a single-stranded RNA. In a further embodiment, the target molecule is mRNA. In a preferred embodiment, the target molecule is Bcl-2 pre-mRNA or Bcl-2 mRNA. In a specific embodiment, the antisense oligomers hybridize to a portion anywhere along a Bcl-2 pre-mRNA or mRNA. The antisense oligomers are preferably selected from those oligomers which hybridize to the translation initiation site, donor splicing site, acceptor splicing site, sites for transportation, or sites for degradation of the Bcl-2 pre-mRNA or mRNA.

In one embodiment, the Bcl-2 antisense oligomer hybridizes to a sequence in the coding region of a Bcl-2 mRNA. In a further embodiment, the oligomer can decrease expression of a Bcl-2 gene product. In another embodiment, the Bcl-2 antisense oligomer hybridizes to a sequence found in a non-coding region of a Bcl-2 mRNA or pre-mRNA, e.g., a sequence found in the upstream regulatory region required for translation of a Bcl-2 message. In a further embodiment, the oligomer can decrease the expression of a Bcl-2 gene product.

In one embodiment, the Bcl-2 antisense oligomer is substantially complementary to a portion of a Bcl-2 pre-mRNA or mRNA, or to a portion of a pre-mRNA or mRNA that is related to Bcl-2. In a further embodiment, the Bcl-2 antisense oligomer hybridizes to a portion of the translation-initiation site of the pre-mRNA coding strand. In another embodiment, the Bcl-2 antisense oligomer hybridizes to a portion of the pre-mRNA coding strand that comprises the translation-initiation site of the human Bcl-2 gene. In yet another embodiment, the Bcl-2 antisense oligomer comprises a TAC sequence which is complementary to the AUG initiation sequence of a Bcl-2 pre-mRNA or RNA.

In another embodiment, the Bcl-2 antisense oligomer hybridizes to a portion of the splice donor site of the pre-mRNA coding strand for the human Bcl-2 gene. Preferably, this nucleotide comprises a CA sequence, which is complementary to the GT splice donor sequence of a Bcl-2 gene, and preferably further comprises flanking portions of 5 to 50 bases, more preferably from about 10 to 20 bases, which hybridizes to portions of a Bcl-2 gene coding strand flanking said splice donor site.

In yet another embodiment, the Bcl-2 antisense oligomer hybridizes to a portion of the splice acceptor site of the pre-mRNA coding strand for the human Bcl-2 gene. Preferably, this nucleotide comprises a TC sequence, which is complementary to the AG splice acceptor sequence of a Bcl-2 gene, and preferably further comprises flanking portions of 5 to 50 bases, more preferably from about 10 to 20 bases, which hybridizes to portions of a Bcl-2 gene coding strand flanking said splice acceptor site. In another embodiment, the Bcl-2 antisense oligomer hybridizes to portions of the pre-mRNA or mRNA involved in splicing, transport or degradation.

One of average skill in the art can recognize that antisense oligomers suitable for use in the invention may also be substantially complementary to other sites along a Bcl-2 pre-mRNA or mRNA, and can form hybrids. The skilled artisan will also appreciate that antisense oligomers that hybridize to a portion of a Bcl-2 pre-mRNA or mRNA, but whose sequence does not commonly occur in transcripts from unrelated genes, are preferable so as to maintain treatment specificity.

Examples of Bcl-2 antisense oligomers that may be used in accordance with the present invention are described in detail in U.S. Pat. No. 5,734,033; U.S. Pat. No. 5,831,066; and U.S. Pat. No. 6,040,181, each of which is incorporated herein by reference in its entirety. A preferred Bcl-2 antisense oligomer comprises the sequence: 5′-TCTCCCAGCGTGCGCCAT-3′ (also known as G3139, oblimersen or Genasense®).

The design of the sequence of a Bcl-2 antisense oligomer can also be determined by empirical testing and assessment of activity in an art-recognized model system or clinical effectiveness, regardless of its degree of sequence homology to, or hybridization with, a Bcl-2 gene, Bcl-2 pre-mRNA, Bcl-2 mRNA, or Bcl-2 related nucleotide sequences. One of ordinary skill in the art will appreciate that Bcl-2 antisense oligomers having, for example, less sequence homology, greater or fewer modified nucleotides, or longer or shorter lengths, compared to those of the preferred embodiments, but which nevertheless demonstrate effectiveness in clinical treatments, are also within the scope of the invention.

The antisense oligomers may be RNA or DNA, or derivatives thereof. The particular form of antisense oligomer may affect the oligomer's pharmacokinetic parameters such as bioavailability, metabolism, half-life, etc. As such, the invention contemplates antisense oligomer derivatives having properties that improve cellular uptake, enhance nuclease resistance, improve binding to the target sequence, or increase cleavage or degradation of the target sequence. The antisense oligomers may contain bases comprising, for example, phosphodiesters, phosphorothioates or methylphosphonates, among others. In one embodiment, the antisense oligomers, instead, can be mixed oligomers. Such oligomers may possess modifications which comprise, but are not limited to, 2-O′-alkyl or 2-O′-halo sugar modifications, backbone modifications (e.g. methylphosphonate, phosphorodithioate, phosphordithioate, formacetal, 3′-thioformacetal, sulfone, sulfamate, nitroxide backbone, morpholino derivatives and peptide nucleic acid (PNA) derivatives), or derivatives wherein the base moieties have been modified (Egholm et al., 1992, Peptide Nucleic Acids (PNA)-Oligomer Analogues With An Achiral Peptide Backbone; Nielsen et al., 1993, “Peptide nucleic acids (PNAs): potential antisense and anti-gone agents”, Anticancer Drug Des 8:53 63). Mixed oligomers may comprise any combination of modified bases. In another embodiment, antisense oligomers comprise conjugates of the oligomers and derivatives thereof (Goodchild, 1990, “Conjugates of oligomers and modified oligomers: a review of their synthesis and properties”, Bioconjug. Chem. 1(3):165 87).

For in vivo therapeutic use, several types of nucleoside derivatives are available. A phosphorothioate derivative of the oligomers of the invention can be useful for in vivo therapeutic use, in part due to the greater resistance to degradation. In one embodiment, the Bcl-2 antisense oligomer comprises phosphorothioate bases. In another embodiment, the Bcl-2 antisense oligomer contains at least one phosphorothioate linkage. In another embodiment, the Bcl-2 antisense oligomer contains at least three phosphorothioate linkages. In a further embodiment, the Bcl-2 antisense oligomer contains at least three consecutive phosphorothioate linkages. In yet another embodiment, the Bcl-2 antisense oligomer is comprised entirely of phosphorothioate linkages. Methods for preparing oligonucleotide derivatives are known in the art.

### Tyrosine Kinase Inhibitors

Examples of tyrosine kinase inhibitors include imatinib (sold under the tradename Gleevec® by Novartis), sunitinib (sold under the tradename Sutent® by Pfizer), sorafenib (sold under the tradename Nexavar® by Bayer Healthcare), gefitinib (sold under the tradename Iressa® by AstraZeneca Pharmaceuticals), and erlotinib (sold under the tradename Tarceva® by Genentech). According to the package inserts, Gleevec targets the following receptors: Bcr-able, PDGF, c-kit; Sutent targets PDGFRα, PDGFRβ, vascular endothelial growth factor receptors (VEGFR1, VEGFR2, VEGFR3), stem cell factor receptor (KIT), fms-like tyrosine kinase-3 (FLT3), colony stimulating factor receptor Type 1 (CSF-1R); Nexavar targets CRAF, BRAF and mutant BRAF, cell surface kinases (KIT, FLT-3, VEGFR-2, VEGFR-3) and PDGFRβ; Iressa targets EGFR and other intracellular kinases; Tarceva targets EGFR and other intracellular kinases. PDGF, c-kit and FLT3 belong to the juxtamembrane family of receptors.

A variety of other tyrosine kinase inhibitors are in development and are expected to display a similar enhanced effect in combination therapy according to the present invention. For example, Telatanib (Bay 57-9352), Axitinib (AG-013736), Dasatinib, KRN951 (Kirin Research Inst.), Vatalanib (PTK787/ZK222584) and E7080 (Esai Pharmaceuticals) have appropriate kinase inhibition profiles for use in the invention. In addition, Table 1 of R. K. Jain, et al. Nature Clinical Practice Oncology (2006) 3, 24-40, incorporated herein by reference, lists additional VEGFR, PDGFR, c-kit and FLT3 inhibitors that would also be useful in the invention.

### Methods of Use of Oligomers, Kinase Inhibitors, and Cancer Therapeutics

In a preferred embodiment, the invention further encompasses the use of combination therapy to prevent or treat cancer. Combination therapy includes the administration of a Bcl-2 antisense oligomer, a tyrosine kinase inhibitor, and the use of one or more molecules, compounds or treatments that aid in the prevention or treatment of cancer, which molecules, compounds or treatments includes, but is not limited to, chemoagents, immunotherapeutics, cancer vaccines, anti-angiogenic agents, cytokines, hormone therapies, gene therapies, and radiotherapies. In the present invention, the length, timing and dosing of a cycle of therapy will be determined by the type of drugs selected. Treatment regimens for tyrosine kinase inhibitors, Bcl-2 antisense oligomers and cancer drugs are known in the art and the skilled artisan can adapt these protocols for use in the present invention without the exercise of inventive skill.

In a further preferred embodiment, one or more cytotoxic chemoagents, in addition to the Bcl-2 antisense oligomer and the tyrosine kinase inhibitor, are administered to treat a cancer patient. Examples of chemoagents contemplated by the present invention include, but are not limited to, cytosine arabinoside, taxoids (e.g., paclitaxel, docetaxel), anti-tubulin agents (e.g., paclitaxel, docetaxel, Epothilone B, or its analogues), cisplatin, carboplatin, adriamycin, tenoposide, mitozantron, 2-chlorodeoxyadenosine, alkylating agents (e.g., cyclophosphamide, mechlorethamine, thioepa, chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin, thio-tepa), antibiotics (e.g., dactinomycin (formerly actinomycin), bleomycin, mithramycin, anthramycin), antimetabolites (e.g., methotrexate, 6-mercaptopurine, 6-thioguanine, cytarabine, 5-fluorouracil, fludarabine, gemcitabine, dacarbazine, temozolamide), asparaginase, Bacillus Calmette and Guerin, diphtheria toxin, hexamethylmelamine, hydroxyurea, LYSODREN®, nucleoside analogues, plant alkaloids (e.g., Taxol, paclitaxel, camptothecin, topotecan, irinotecan (CAMPTOSAR, CPT-11), vincristine, vinca alkyloids such as vinblastine), podophyllotoxin (including derivatives such as epipodophyllotoxin, VP-16 (etoposide), VM-26 (teniposide)), cytochalasin B, gramicidin D, ethidium bromide, emetine, mitomycin, procarbazine, mechlorethamine, anthracyclines (e.g., daunorubicin (formerly daunomycin), doxorubicin, doxorubicin liposomal), dihydroxyanthracindione, mitoxantrone, mithramycin, actinomycin D, procaine, tetracaine, lidocaine, propranolol, puromycin, anti-mitotic agents, abrin, ricin A, pseudomonas exotoxin, nerve growth factor, platelet derived growth factor, tissue plasminogen activator, aldesleukin, allutamine, anastrozle, bicalutamide, biaomycin, busulfan, capecitabine, carboplain, chlorabusil, cladribine, cylarabine, daclinomycin, estramusine, floxuridhe, gamcitabine, gosereine, idarubicin, itosfamide, lauprolide acetate, levamisole, lomusline, mechlorethamine, magestrol, acetate, mercaptopurino, mesna, mitolanc, pegaspergase, pentoslatin, picamycin, riuxlmab, campath-1, straplozocin, thioguanine, tretinoin, vinorelbine, or any fragments, family members, or derivatives thereof, including pharmaceutically acceptable salts thereof. Compositions comprising one or more chemoagents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone.

### EXAMPLES

Genasense® (G3139 by Genta, Bcl-2 inhibitor, also referred to in the Figures as “OBL”), Abraxane® (by Abraxis, paclitaxel), and Taxol® (by Bristol Myers Squibb, paclitaxel) were submitted for evaluation in combination with tyrosine kinase inhibitors against an A549 human lung cancer model in severe combined immune deficient (SCID) mice. Ten treatment groups having 10 mice per group were analyzed for tumor growth delay. G3139, Abraxane, and Taxol were administered via intravenous (IV) injection, and tyrosine kinase inhibitors were administered via oral gavage (PO).

The mice were injected with 100 μl tumor cells subcutaneously (SC). The tumors were allowed to grow for 14 days. On the 15th day, treatment cycles of 5 days on and 2 days off began for a series of 3 cycles. During treatment, the mice were weighed twice daily. Tumor volume estimation (mm³) was made in accordance with the formula (a²×b/2), where “a” is the smallest diameter and “b” is the largest diameter. All procedures were completed in accordance with established animal care protocols.

The A549 human lung cancer cell line was grown in HyQ RPMI-1640 (1×) media (HyClone, Logan, Utah) supplemented with 10% fetal bovine serum (Sigma, St. Louis, Mo.) and maintained in 5% CO₂-95% air humidified atmosphere at 37° C. Subconfluent cells were harvested by using 0.23% trypsin-EDTA (HyClone, Logan, Utah) and were counted using the trypan blue assay technique. Cells (99% viability) were re-suspended at the concentration of 10×10⁶ cells/100 μl of sterile saline.

G3139 was prepared fresh each treatment day from a refrigerated stock solution of 30 mg/ml using sterile saline as the vehicle; while tyrosine kinase inhibitors in capsule form were brought up to the desired concentration using 10% DMSO and 90% sterile saline as the vehicle. Taxol (Infusion Solutions, Tucson, Az.) was prepared fresh each treatment day from a refrigerated stock solution of 6 mg/ml in Cremaphor EL/EtOH using sterile saline as the vehicle. Abraxane was prepared from a frozen stock of 5 mg/ml with the diluent of sterile saline. G3139 (20 mg/kg) was administered twice during each 5-day treatment cycle, on day 3 and on day 5. Abraxane and Taxol were administered once on day 1 via IV injection (13.4 mg/kg). G3139 was administered at a volume of 0.1 ml using a 27 gauge needle (Becton Dickinson). Abraxane and Taxol were administered at a volume of 0.2 ml using a 27 gauge needle. Tyrosine kinase inhibitors were administered daily during the 5 day treatment cycle. Both Gleevec and Tarceva were administered at a rate of 100 mg/kg, and both Sutent and Nexavar were administered at a rate of 40 mg/kg. A negative control comprising the delivery vehicle only was also administered.

### Example 1

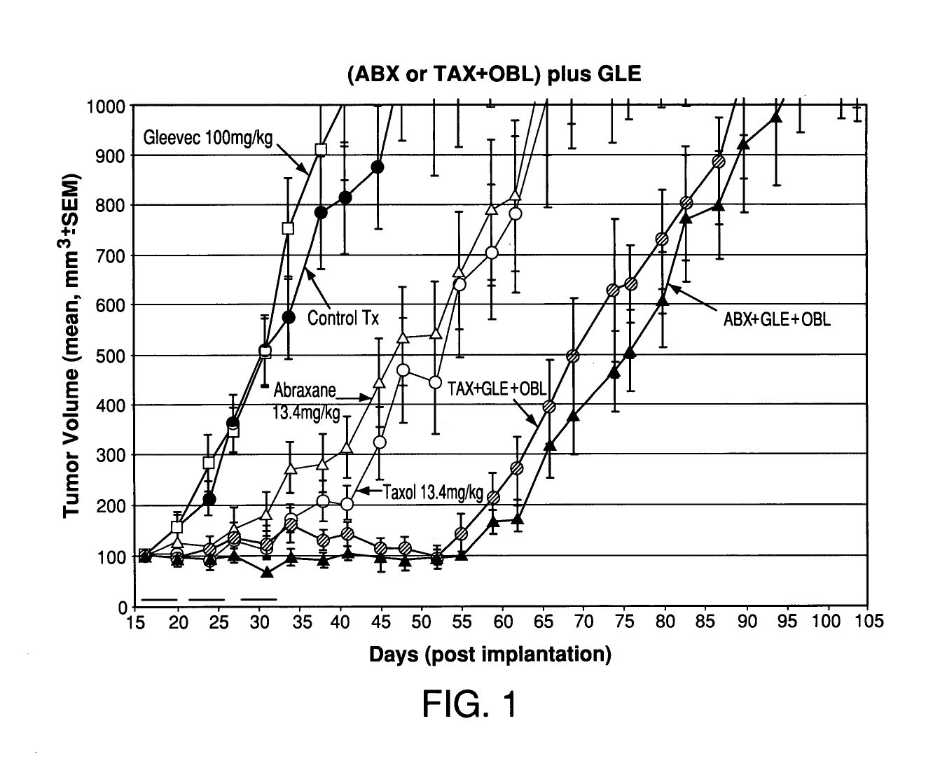

In accordance with the treatment cycle described above, certain treatment regimens were administered: Gleevec alone; Gleevec combined with Genasense (OBL) and Taxol; and Gleevec combined with Genasense (OBL) and Abraxane. FIG. 1 shows tumor growth (mean, mm³+/−SEM) versus days (post implantation). Gleevec alone and the negative control treatment showed similar tumor growth. Tumor growth after treatment with Taxol alone or with Abraxane alone showed a substantially similar and minor inhibition of tumor growth. Genasense, based on data from other experiments, has a similar minor single-agent activity. Both triplet regimens of Gleevec combined with Genasense and Taxol and of Gleevec combined with Genasense and Abraxane delayed tumor growth for approximately 50-55 days compared to the other regimens, which showed no delay in tumor growth after completion of treatment. Although tumor growth was substantially delayed, the rate of growth for the triplet regimen involving Gleevec was similar to that of Taxol alone and Abraxane alone once growth was initiated.

These results demonstrate the unexpectedly enhanced interaction of the combination treatment of Genasense, Taxol/Abraxane and Gleevec. In view of the fact that Gleevec had no effect on tumor growth, and that Taxol, Abraxane and Genasense each had nearly identical and minimal single-agent effect, it would be predicted that the triplet therapy would be no more efficacious than Taxol, Abraxane or Genasense alone. In contrast, the triplet therapy suppressed tumor growth substantially compared to the single-agents.

### Example 2

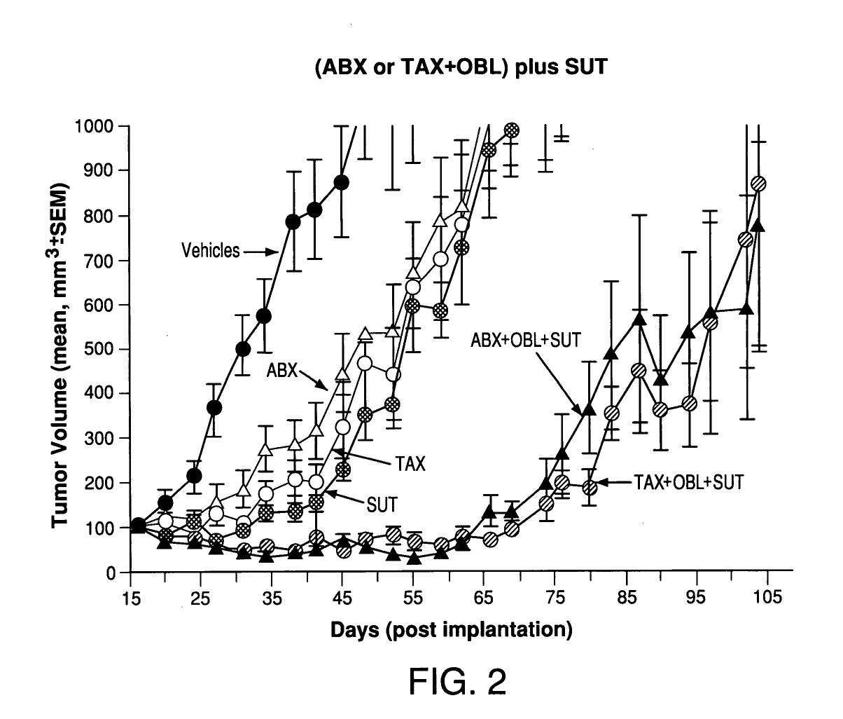

In accordance with the treatment cycle described above, certain treatment regimens were administered: Sutent alone; Sutent combined with Genasense (OBL) and Taxol; and Sutent combined with Genasense (OBL) and Abraxane. FIG. 2 shows tumor growth (mean, mm³+/−SEM) versus days (post implantation). Sutent alone, Taxol alone and Abraxane alone each showed similar tumor growth, and only minor growth inhibition compared to the negative control. Both triplet regimens of Sutent combined with Genasense and Taxol and of Sutent combined with Genasense and Abraxane showed substantially delayed tumor growth (60-65 days) and a substantially reduced rate of growth compared to the single-agent regimens once tumor growth began.

These results demonstrate the unexpectedly enhanced interaction of the combination treatment of Genasense, Taxol/Abraxane and Sutent. In view of the fact that Sutent, Taxol, Abraxane and Genasense each had nearly identical and minimal single-agent effect, it would be predicted that the triplet therapy would be no more efficacious than Sutent, Taxol, Abraxane or Genasense alone. In contrast, the triplet therapy suppressed tumor growth substantially compared to the single-agents. Sutent appears to be more effective in the triplet therapy than Gleevec.

### Example 3

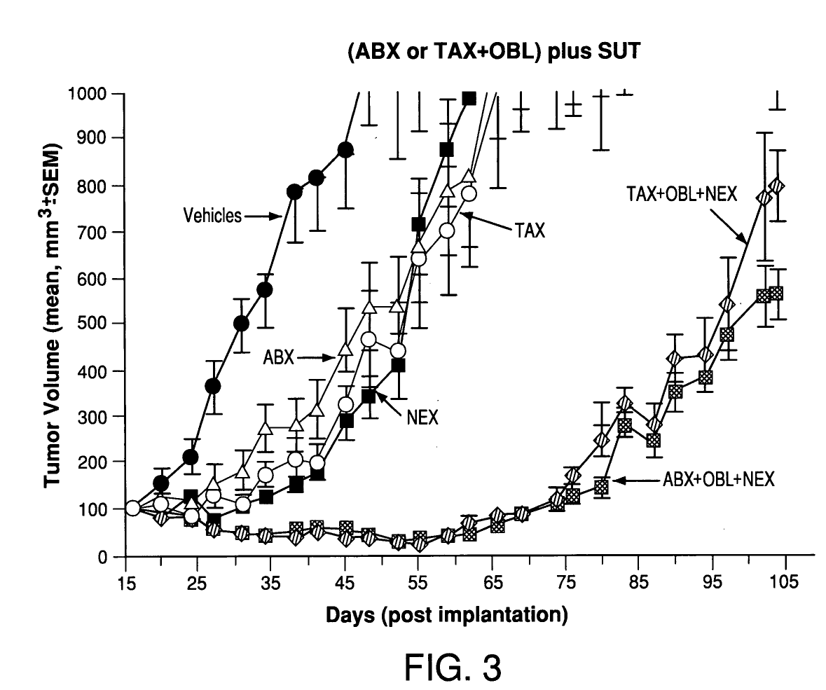

In accordance with the treatment cycle described above, certain treatment regimens were administered: Nexavar alone; Nexavar combined with Genasense (OBL) and Taxol; and Nexavar combined with Genasense (OBL) and Abraxane. FIG. 3 shows tumor growth (mean, mm³+/−SEM) versus days (post implantation). Nexavar alone, Taxol alone and Abraxane alone each showed minor and substantially similar tumor growth inhibition compared to the negative control. Both triplet regimens of Nexavar combined with Genasense and Taxol and of Nexavar combined with Genasense and Abraxane showed substantially delayed tumor growth (70-75 days) and a substantially reduced rate of growth compared to the single-agent regimens once tumor growth began.

These results demonstrate the unexpectedly enhanced interaction of the combination treatment of Genasense, Taxol/Abraxane and Nexavar. In view of the fact that Nexavar, Taxol, Abraxane and Genasense each had nearly identical and minimal single-agent effect, it would be predicted that the triplet therapy would be no more efficacious than Nexavar, Taxol, Abraxane or Genasense alone. In contrast, the triplet therapy suppressed tumor growth substantially compared to the single-agents. Nexavar appears to be comparable to Sutent in the triplet therapy and more effective than Gleevec.

### Example 4

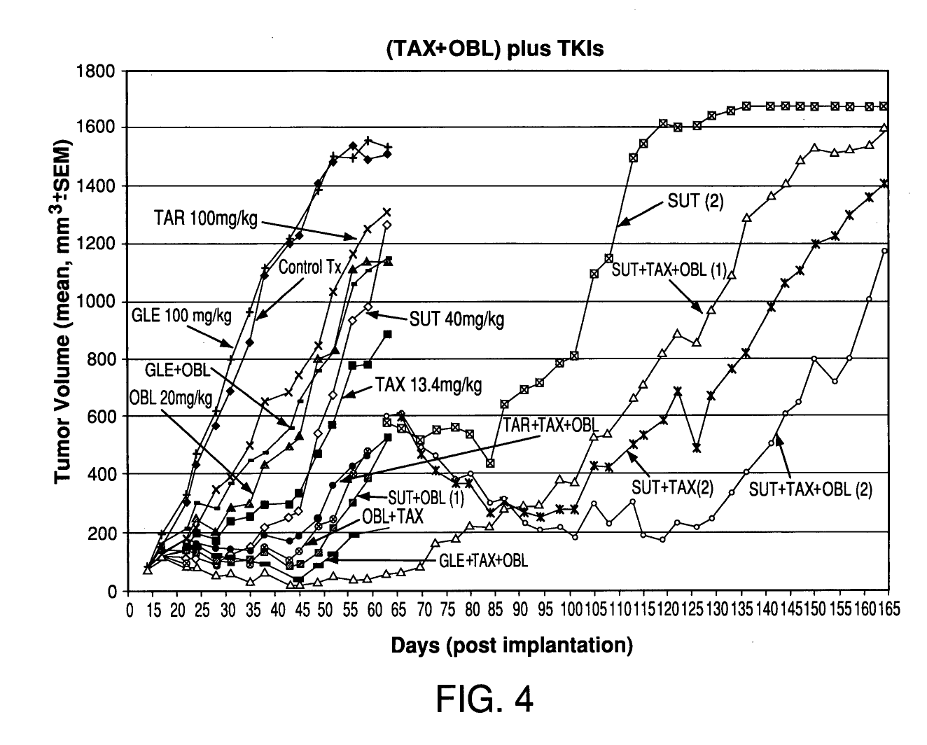

FIG. 4 shows various treatment regimens including Taxol alone, Genasense (OBL) alone, Tarceva alone, Gleevec alone, Sutent alone; doublet of Taxol and Genasense (OBL), doublet of Gleevec and Genasense (OBL), doublet of Sutent and Genasense (OBL); triplet of Taxol, Genasense (OBL), and Tarceva, triplet of Taxol, Genasense (OBL), and Gleevec, and triplet of Taxol, Genasense (OBL), and Sutent. For the triplet of Taxol, Genasense (OBL), and Sutent, the treatment was repeated in mice that had developed tumors of about 600 mm³ following the first treatment regimen.

FIG. 4 shows that Tarceva alone and Genasense alone resulted in similar tumor growth inhibition, which was minor compared to the negative control. The triplet of Taxol, Genasense, and Tarceva was no more effective than the doublet of Taxol and Genasense, showing a similar delay in growth and regrowth rate. However, the triplet of Taxol, Genasense, and Sutent resulted in a prolonged delay in tumor growth (about 70 days) as well as a substantially reduced rate of growth once tumor growth began as compared to all other dosing regimens.

These results confirm the unexpectedly enhanced interaction of the combination treatment of Genasense, Taxol/Abraxane and Sutent found in Example 2. In contrast, however, Tarceva, Taxol, and Genasense each had similar and minimal single-agent effect, but the enhanced interaction in the triplet therapy was not observed.

### Conclusions

Sutent, Nexavar and Gleevec exhibit an unexpectedly enhanced interaction with Bcl-2 antisense and cytotoxic chemotherapeutics in triplet therapy. In contrast, the tyrosine kinase inhibitor Tarceva does not significantly enhance the efficacy of Bcl-2 antisense and cytotoxic chemotherapeutics in triplet therapy. Sutent and Nexavar reportedly inhibit the cell surface kinase receptor families PDGFR, VEGFR, KIT and FLT-3. Gleevec reportedly inhibits PDGFR and c-kit. Tarceva does not target any of these tyrosine kinases, but instead inhibits EGFR and other intracellular kinases. It is therefore believed that the combination of Bcl-2 antisense, a cytotoxic chemotherapeutic and a tyrosine kinase inhibitor that targets a cell surface kinase receptor such as VEGFR, KIT, PDGFR and/or FLT-3 (or a subcombination thereof) provides an unexpectedly enhanced and efficacious treatment for cancer which can substantially delay the growth of tumors and decrease tumor growth rate.

A variety of additional tyrosine kinase inhibitors that fit this profile of kinase inhibition are in development and are expected to display a similar enhanced effect in combination therapy according to the invention. For example, Telatanib (Bay 57-9352), Axitinib (AG-013736), Dasatinib, KRN951 (Kirin Research Inst.), Vatalanib (PTK787/ZK222584) and E7080 (Esai Pharmaceuticals) have appropriate kinase inhibition profiles. In addition, Table 1 of R. K. Jain, et al. Nature Clinical Practice Oncology (2006) 3, 24-40 lists additional VEGFR, PDGFR, c-kit and FLT3 inhibitors that would also be useful in the invention.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A method of inhibiting the growth of cancer cells comprising:
contacting the cancer cells with a Bcl-2 antisense oligomer;
contacting the cancer cells with a tyrosine kinase inhibitor; and
contacting the cancer cells with a cytotoxic chemotherapeutic agent.

2. The method of claim 1, wherein the Bcl-2 antisense oligomer comprises an oblimersen compound.

3. The method of claim 1, wherein the tyrosine kinase inhibitor targets a cell surface kinase receptor.

4. The method of claim 3, wherein the cell surface kinase receptor comprises a vascular endothelial growth factor receptor, a stem cell factor receptor, or an fms-like tyrosine kinase-3 receptor.

5. The method of claim 1, wherein the cytotoxic chemotherapeutic agent comprises dacarbazine, docetaxel, paclitaxel, cisplatin, 5-fluorouracil, doxorubicin, etoposide, cyclophosphamide, fludarabine, irinotecan, or cytosine arabinoside (Ara-C).

6. A method of treating cancer in a human comprising administering to the human a Bcl-2 antisense oligomer, a tyrosine kinase inhibitor, and a cytotoxic chemotherapeutic agent.

7. The method of claim 6, wherein the Bcl-2 antisense oligomer is administered twice per week, the tyrosine kinase inhibitor is administered five times per week, and the cytotoxic chemotherapeutic agent is administered once per week.

8. The method of claim 6, wherein the Bcl-2 antisense oligomer comprises an oblimersen compound; the tyrosine kinase inhibitor comprises sunitinib, sorafenib, or combinations thereof; and the cytotoxic chemotherapeutic agent comprises paclitaxel.

9. The method of claim 6, wherein the Bcl-2 antisense oligomer is administered before the cytotoxic chemotherapeutic agent.

10. A kit comprising a Bcl-2 antisense oligomer in an amount sufficient for at least one cycle of cancer treatment, a tyrosine kinase inhibitor in an amount sufficient for at least one cycle of cancer treatment, and a cytotoxic chemotherapeutic agent in an amount sufficient for at least one cycle of cancer treatment.

11. The kit of claim 10, wherein the cycle is a five-day cycle.
